(19) United States
(12) Patent Application Publication
(10) Pub. No.: US 2020/0078290 A1
(43) Pub. Date: Mar. 12, 2020

(54) Cosmetic Composition Comprising Extract Of Medicinal Herbs As Active Ingredient

(71) Applicant: LG Household & Health Care Ltd., Seoul (KR)

(72) Inventors: Byunghyun Kim, Deajeon (KR); Hyosun Roh, Daejeon (KR); Cho-Young Hong, Daejeon (KR); Young Je Ahn, Daejeon (KR); Seung-Jin Hwang, Daejeon (KR); Mu Hyun Jin, Daejeon (KR)

(73) Assignee: LG Household & Health Care Ltd., Seoul (KR)

(21) Appl. No.: 16/470,044

(22) PCT Filed: Dec. 15, 2017
(86) PCT No.: PCT/KR2017/014863

§ 371 Date: Jun. 14, 2019

(30) Foreign Application Priority Data
Dec. 15, 2016 (KR) 10-2016-0171795
Dec. 15, 2016 (KR) 10-2016-0171796
Dec. 15, 2016 (KR) 10-2016-0171797

(51) Int. Cl. A61K 8/97; A61K 8/04; A61K 8/06; A61Q 19/08

(52) U.S. Cl. CPC A61K 8/97; A61K 8/046; A23L 33/105; A61Q 19/08; A61K 2800/59; A61K 8/06

(57) ABSTRACT

A cosmetic composition comprising a Dimocarpus longan Fruit, Vitex trifolia Fruit and/or Aquilaria agallocha Stem extract as an active ingredient. The cosmetic composition has excellent effects in enhancing skin elasticity, improving wrinkles, moisturizing skin or improving skin troubles. Further, food composition and a quasi-drug composition comprising a Dimocarpus longan Fruit, Vitex trifolia Fruit and/or Aquilaria agallocha Stem extract as an active ingredient are provided.

### TECHNICAL FIELD

The present invention relates to a cosmetic composition including a Dimocarpus longan Fruit, Vitex trifolia Fruit, and/or Aquilaria agallocha Stem extract as an active ingredient. More specifically, the present invention relates to a cosmetic composition having excellent effects in enhancing skin elasticity, improving wrinkles, moisturizing skin, or improving skin troubles. Further, the present invention relates to a food composition and a quasi-drug composition including a Dimocarpus longan Fruit, Vitex trifolia Fruit, and/or Aquilaria agallocha Stem extract as active ingredients.

### BACKGROUND ART

Collagen is a major substrate protein produced in the fibroblasts of the skin and is present in the extracellular matrix. It is known that the collagen serves important functions of mechanical durability of the skin, resistance of connective tissues and binding force of tissues, support of cell adhesion, induction of cell division and differentiation (during growth of organisms or wound healing). Such collagen is reduced with age and with photoaging due to ultraviolet irradiation, and collagen reduction is stimulated by collagenase activity, which degrades collagen. This is known to be closely related to the formation of wrinkles in the skin.

In addition, elastin fibers form crosslinks with collagen and are important skin components in wrinkle formation involved in skin elasticity. The deficiency and aggregation of elastin fibers, and a dramatic increase in the activity of elastase, which is an elastin-degrading enzyme, have been found to be one of the causes of formation of skin wrinkles. Elastase is the only enzyme capable of degrading elastin, and it is known that the inhibition of elastase can fundamentally reduce the improvement of skin wrinkles.

Moreover, collagen and elastin fibers are substrate proteins that play an important role in moisture retention in the dermis. These substrate proteins adsorb moisture and increase the moisture retention power inside the structure that the proteins form, allowing the skin to maintain moisture at an appropriate level, and are also known to be involved in maintaining elasticity of the skin through moisture.

Currently, retinoids, adenosine, animal placenta-derived proteins, chlorella extracts, and the like are known as cosmetics for enhancing skin elasticity and improving wrinkles. The most well-known retinol is a substance that promotes collagen synthesis and inhibits elastase, but is unstable and its amount of use is restricted due to safety problems, such as irritation and redness, when applied to the skin. Further, the chlorella extract and the like have little effect, and thus, it is substantially difficult to expect skin elasticity enhancement and wrinkle improvement effects.

Cosmetic compositions for moisturization generally used for the skin provides a soft and vivid look and functions to prevent damages such as cracks, dryness or the like by maintaining a certain amount of moisture on human hair or skin. That is, the cosmetic compositions for moisturizing skin are used for the purpose of beautifying the skin or hair and keeping them healthy, by supplying more than a certain amount of moisture to the skin or hair or maintaining the moisture.

Since the skin is responsible for about 65% to 70% of moisture control, studies on the supply, maintenance, that is, moisturization, are actively conducted in the field of cosmetics. Recently, cosmetics that increase moisturizing power by introducing physiologically active substances obtained from natural substances or the like into cosmetics in order to maintain the inherent defensive function of the skin itself have been actively developed.

For example, Korean Patent Application Publication No. 2002-0044266 discloses a cosmetic composition for skin protection, which includes a complex herbal extract. However, it does not disclose the effect of the herbal extract alone and the skin protection effect thereof.

Cosmetics are products used to protect the skin and for beautification and cleanliness of the skin, but the composition thereof inevitably includes ingredients that are different from the purpose of skin protection in order to form the products. These ingredients include surfactants, preservatives, fragrances, sunscreen blockers, colorings, as well as various ingredients for imparting other efficacy and effects. These ingredients are generally known to cause various skin troubles such as inflammation, rash, edema, and the like on the skin (Maibach. H. I., Contact Dermatitis, 6. 369-404, 1980). In addition, sebum and sweat excreted from the body, fatty acids in cosmetic ingredients, higher alcohols, protein components and the like may be degraded into highly toxic substances by skin pathogens existing on the skin and cause skin inflammation. Further, it is well known that skin inflammation is caused by ultraviolet rays from the sun.

Inflammation reactions are manifested by five phenomena, such as feeling of redness, pricking feeling, burning hotness, swelling, and change in tissues, and there are physiological reactions to protect the living body from harmful environment, that is, from invasion of external substances such as bacteria and mechanical damages. These inflammatory phenomena lead to a dramatic increase in various types of polymorphonuclear leukocytes (PMNs) and immune substances, and these increased cells secrete various types of proteases and cytokines, which are inflammatory cell products, thereby allowing for treatment and defense. However, these actions may also cause harmful damage to adjacent tissue cells and non-cellular components. Therefore, under proper conditions, the normal function is restored after the initial state of inflammation, but when irritants stimulating the inflammation are not eliminated or continuously produced, chronic inflammation occurs ultimately, resulting in more serious tissue damage.

Meanwhile, Dimocarpus longan Fruit is the arillus of Dimocarpus longan Loureiro (Sapindaceae). Specifically, Dimocarpus longan Fruit is an evergreen tree of Sapindaceae and refers to the pulp of Dimocarpus longan Loureiro, which is the fruit. That is, it is a transparent arillus which appears to be pulpy, and when dried, it becomes black brown Lonaganae arillus. Dimocarpus longan Fruit is mainly distributed and grows in China and Southeast Asia, has a calm, stomach-protecting, nourishing effects and is effective for insomnia. It may be named as gye-won, gyo-lu, mil-bi, al-yeo-ji, yeo-ju, yeon-lan, yong-mok, yong-an, yong-an-geon, won-an, won-an-yuk, ig-ji,cheon-tan-ja, and yeo-ji-no in addition to Dimocarpus longan Fruit. Vitex trifolia Fruit is a medicinal product made from the fruit of Vitex rotundifolia L. of Verbena officinalis. Vitex trifolia Fruit is used for dizziness caused by cold, headache, hypertension, and it is round-shaped or spherical-shaped. The outer surface is black or dark brown, and it is covered with thin grayish white calyxes at the bottom. It is light and hard and does not break easily. It can be named as, so-hyeong, Hyeong-ja, man-hyeong-sil, and man-cheong-ja in addition to Vitex trifolia Fruit.

Aquilaria agallocha Stem is a tree trunk in which the resin of Aquilaria agallocha Roxburgh (Thymeleaceae) is deposited. It is mainly produced in India, Malaysia and southern part of China. Its surface is brown or dark brown with intersection of yellowish segregation and is slightly glossy. Aquilaria agallocha Stem is a part where it appears as a histologically hard lump on the heartwood of Aquilaria agallocha Roxburgh by the decomposition of the resin naturally secreted on Aquilaria agallocha Roxburgh. It may be named as chim-su-hyang, ga-nam-hyang, gi-nam-hyang, mil-hyang, and jan-hyang.

### DISCLOSURE

### Technical Problem

The present inventors have conducted studies on substances having superior skin elasticity-enhancing, wrinkle-improving, skin moisturizing and skin trouble-improving effects from natural resources, and accordingly, they have discovered that the composition including a Dimocarpus longan Fruit, Vitex trifolia Fruit and/or Aquilaria agallocha Stem extract as an active ingredient shows a collagen synthesis-promoting, elastase activity-inhibiting, moisturizing and anti-inflammatory effect, and thus have completed the cosmetic composition, food composition, and quasi-drug composition using the same.

### Technical Solution

It is one object of the present invention to provide a cosmetic composition including one or more extracts selected from the group consisting of Dimocarpus longan Fruit, Vitex trifolia Fruit and Aquilaria agallocha Stem, or a fraction thereof, as an active ingredient.

Specifically, it is one object of the present invention to provide a cosmetic composition for enhancing skin elasticity or improving wrinkles including one or more extracts selected from the group consisting of Dimocarpus longan Fruit, Vitex trifolia Fruit and Aquilaria agallocha Stem, or a fraction thereof, as an active ingredient.

Specifically, it is one object of the present invention to provide a cosmetic composition for moisturizing skin including one or more extracts selected from the group consisting of Dimocarpus longan Fruit, Vitex trifolia Fruit and Aquilaria agallocha Stem, or a fraction thereof, as an active ingredient.

Specifically, it is one object of the present invention to provide a cosmetic composition for improving skin troubles including one or more extracts selected from the group consisting of Dimocarpus longan Fruit, Vitex trifolia Fruit and Aquilaria agallocha Stem, or a fraction thereof, as an active ingredient.

It is another object of the present invention to provide a food composition including one or more extracts selected from the group consisting of Dimocarpus longan Fruit, Vitex trifolia Fruit and Aquilaria agallocha Stem, or a fraction thereof, as an active ingredient.

It is still another object of the present invention to provide a quasi-drug composition including one or more extracts selected from the group consisting of Dimocarpus longan Fruit, Vitex trifolia Fruit and Aquilaria agallocha Stem, or a fraction thereof, as an active ingredient.

### Advantageous Effects

The cosmetic composition of the present invention, which includes a Dimocarpus longan Fruit, Vitex trifolia Fruit and/or Aquilaria agallocha Stem extract as an active ingredient, has excellent effects in enhancing skin elasticity, improving wrinkles, moisturizing skin or improving skin troubles. Specifically, the composition of the present invention has excellent effects in promoting collagen synthesis and inhibiting elastase activity, and has excellent anti-inflammatory and moisturizing effects and thus can be highly utilized as a cosmetic composition for enhancing skin elasticity or improving wrinkles, moisturizing skin and improving skin troubles. In addition, the composition including a Dimocarpus longan Fruit, Vitex trifolia Fruit and Aquilaria agallocha Stem extract, or a fraction thereof as an active ingredient can be used as a food composition and a quasi-drug composition.

### DETAILED DESCRIPTION OF THE EMBODIMENTS

In one aspect, the present invention provides a cosmetic composition including one or more extracts selected from the group consisting of Dimocarpus longan Fruit, Vitex trifolia Fruit and Aquilaria agallocha Stem, or a fraction thereof, as an active ingredient.

As used herein, “Dimocarpus longan Fruit” is the arillus of Dimocarpus longan Loureiro (Sapindaceae). Specifically, Dimocarpus longan Fruit is an evergreen tree of Sapindaceae and refers to the pulp of Dimocarpus longan Loureiro, which is the fruit. That is, it is a transparent arillus which appears to be pulpy, and when dried, it becomes black brown Lonaganae rillus. Dimocarpus longan Fruit is mainly distributed and grows in China and Southeast Asia, has a calm, stomach-protecting, nourishing effects and is effective for insomnia. It may be named as gye-won, gyo-lu, mil-bi, al-yeo-ji, yeo-ju, yeon-lan, yong-mok, yong-an, yong-an-geon, won-an, won-an-yuk, ig-ji,cheon-tan-ja, and yeo-ji-no in addition to Dimocarpus longan Fruit.

As used herein, “Vitex trifolia Fruit” is a medicinal product made from the fruit of Vitex rotundifolia L. of Verbena officinalis. Vitex trifolia Fruit is used for dizziness caused by cold, headache, hypertension, and it is round-shaped or spherical-shaped. The outer surface is black or dark brown, and it is covered with thin grayish white calyxes at the bottom. It is light and hard and does not break easily. It can be named as, so-hyeong, Hyeong-ja, man-hyeong-sil, and man-cheong-ja in addition to Vitex trifolia Fruit.

As used herein, “Aquilaria agallocha Stem” is a tree trunk in which the resin of Aquilaria agallocha Roxburgh (Thymeleaceae) is deposited. It is mainly produced in India, Malaysia and southern part of China. Its surface is brown or dark brown with intersection of yellowish segregation and is slightly glossy. Aquilaria agallocha Stem is a part where it appears as a histologically hard lump on the heartwood of Aquilaria agallocha Roxburgh by the decomposition of the resin naturally secreted on Aquilaria agallocha Roxburgh. It may be named as chim-su-hyang, ga-nam-hyang, gi-nam-hyang, mil-hyang, and jan-hyang.

As used herein, the term “extract” includes an extract liquid itself and extracts of all formulations which can be formed using the extract liquid, such as an extract liquid obtained by extracting the Dimocarpus longan Fruit, Vitex trifolia Fruit or Aquilaria agallocha Stem, a diluted solution or concentrate of the extract liquid, a dried product obtained by drying the extract liquid, a crude-purified or purified product of the extract liquid, or mixtures thereof.

The extract of the present invention may be extracted from natural, hybrid, or mutant plants of the respective plants, and can also be extracted from plant tissue cultures.

In the extraction of the Dimocarpus longan Fruit, Vitex trifolia Fruit and Aquilaria agallocha Stem of the present invention, a method for extracting the extracts is not particularly limited, and extraction can be performed according to methods commonly used in the art. Non-limiting examples of the extraction method include hydrothermal extraction method, ultrasonic extraction method, filtration method, and reflux extraction method and the like. These methods may be carried out alone or in combination of two or more thereof.

In the present invention, the type of extraction solvent used for extracting the Dimocarpus longan Fruit, Vitex trifolia Fruit and Aquilaria agallocha Stem is not particularly limited, and any solvent known in the art may be used. Non-limiting examples of the extraction solvent include water; C₁ to C₄ lower alcohols such as methanol, ethanol, propyl alcohol, butyl alcohol and the like; polyhydric alcohols such as glycerin, butylene glycol, propylene glycol and the like; and hydrocarbon-based solvents such as methyl acetate, ethyl acetate, acetone, benzene, hexane, diethyl ether, dichloromethane and the like; or mixtures thereof. Preferably, water, lower alcohols, 1,3-butylene glycol, and ethyl acetate may be used alone or in a mixture of two or more thereof.

In the present invention, water (or distilled water) may be more specifically used as a solvent for extracting the medicinal herbs of Dimocarpus longan Fruit, Vitex trifolia Fruit or Aquilaria agallocha Stem. The above-mentioned solvents can be used to prepare a solvent extract by extracting the medicinal herbs of Dimocarpus longan Fruit, Vitex trifolia Fruit or Aquilaria agallocha Stem more than once, and a dried extract, which is obtained by subjecting the solvent extract to vacuum distillation, followed by freeze-drying or spray drying, can be prepared. In one specific embodiment of the present invention, water (or distilled water) was used to prepare each of the Dimocarpus longan Fruit, Vitex trifolia Fruit and Aquilaria agallocha Stem extracts.

In the present invention, an extract obtained by hydrothermal extraction or cold-water extraction may be used as it is after filtration to remove suspended solid particles using, for example, nylon or the like to filter particles, or filtration using freeze filtration or the like, or it may be used by drying through freeze drying, hot air drying, spray drying or the like.

As used herein, the term “fraction” refers to a resulting product obtained by performing fractionation to separate a specific component or a group of specific components from a mixture containing various different constituent components.

In the present invention, a fractionation method for obtaining the fraction is not particularly limited, and fractionation can be performed according to methods commonly used in the art. Non-limiting examples of the fractionation method include a method of obtaining a fraction from the extract, which has been obtained by extracting Dimocarpus longan Fruit, Vitex trifolia Fruit and Aquilaria agallocha Stem, by treating the extract with a predetermined solvent.

In the present invention, the type of fractionation solvent used for obtaining the fraction is not particularly limited, and any solvent known in the art can be used. Non-limiting examples of the fractionation solvent include polar solvents such as water, alcohol, and the like; and non-polar solvents such as hexane, ethyl acetate, chloroform, dichloromethane, and the like. These may be used alone or in combination of one or more thereof. When an alcohol is used in the fraction solvent, C₁ to C₄ alcohols can be specifically used.

In one or more extracts selected from the group consisting of Dimocarpus longan Fruit, Vitex trifolia Fruit and Aquilaria agallocha Stem of the present invention, two or more mixed extracts may be prepared by extracting each of the Dimocarpus longan Fruit, Vitex trifolia Fruit and Aquilaria agallocha Stem and then mixing two or more extracts, or may be prepared by extracting two or more selected from the group consisting of Dimocarpus longan Fruit, Vitex trifolia Fruit and Aquilaria agallocha Stem together.

In two or more extracts selected from the group consisting of the Dimocarpus longan Fruit, Vitex trifolia Fruit and Aquilaria agallocha Stem, the weight ratio of two or more selected from the group consisting of the two or more mixed extracts or fractions thereof is not particularly limited, but in case of two mixed extracts, the weight ratio may be specifically 1:1, in case of three mixed extracts, the weight ratio may be 1:1:1.

Specifically, the present invention may provide a cosmetic composition for enhancing skin elasticity or improving wrinkles including one or more extracts selected from the group consisting of Dimocarpus longan Fruit, Vitex trifolia Fruit and Aquilaria agallocha Stem, or a fraction thereof, as an active ingredient.

In the present invention, the term “skin elasticity enhancement” refers to alleviating the degree of skin sagging or drooping, and maintaining skin elasticity in a state where elastin and collagen are present sufficiently as elastic fibers composed of elastin are present along with collagen fibers called collagen. In the present invention, the term “wrinkle improvement” refers to inhibiting or suppressing the generation of wrinkles on the skin, or alleviating wrinkles that have already been formed.

In one embodiment of the present invention, when one or more extracts selected from the group consisting of Dimocarpus longan Fruit, Vitex trifolia Fruit and Aquilaria agallocha Stem were added to a culture medium of human fibroblasts to assess the collagen synthesis promoting effect, all of the experimental groups to which the extract was added showed superior collagen synthesis effect compared to the control group. Further, the collagen synthesis effect was further enhanced when two mixed extracts were added compared to the case where a single extract was treated, and in particular, the collagen synthesis effect was the highest when all three mixed extracts of Dimocarpus longan Fruit, Vitex trifolia Fruit and Aquilaria agallocha Stem were treated (Table 3).

In addition, in one embodiment of the present invention, all of the experimental groups to which one or more extracts selected from the group consisting of Dimocarpus longan Fruit, Vitex trifolia Fruit and Aquilaria agallocha Stem were added showed superior elastase inhibitory effect compared to the control group, and the elastase inhibitory effect was further enhanced when two mixed extracts were added compared to the case where a single extract was treated, and the elastase inhibitory effect was the highest when three mixed extracts were treated (Table 4).

Each extract of Dimocarpus longan Fruit, Vitex trifolia Fruit and Aquilaria agallocha Stem has a collagen synthesis effect and an elastase inhibitory effect, and thus can be used for enhancing skin elasticity or improving wrinkles, and in particular, a more superior effect is observed when two or more mixed extracts are used.

Specifically, the present invention may provide a cosmetic composition for moisturizing skin including one or more extracts selected from the group consisting of Dimocarpus longan Fruit, Vitex trifolia Fruit and Aquilaria agallocha Stem, or a fraction thereof, as an active ingredient.

As used herein, the term “skin moisturization” refers to increasing moisturizing feeling on the skin and maintaining a moist state. Skin moisturizing effect can help to improve wrinkles and enhance elasticity of the skin. One or more extracts selected from the group consisting of Dimocarpus longan Fruit, Vitex trifolia Fruit and Aquilaria agallocha Stem of the present invention showed excellent collagen synthesis effect and elastase activity inhibitory effect, and thus, it was confirmed therefrom that the cosmetic composition of the present invention can be used for moisturizing skin (Tables 3 and 4).

In one embodiment, the moisture retention effect of one or more extracts selected from the group consisting of Dimocarpus longan Fruit, Vitex trifolia Fruit and Aquilaria agallocha Stem of the present invention was confirmed, and as a result, all the experimental groups to which the extract was added exhibited a superior skin moisturizing effect compared to the control group, and when two mixed extracts were added, the water retention effect was further enhanced compared to the case where a single extract was treated, and in particular, the highest water retention effect was obtained when three mixed extracts were treated (Table 5).

The Dimocarpus longan Fruit, Vitex trifolia Fruit and Aquilaria agallocha Stem extracts of the present invention have a moisture retention effect when treated alone and thus can be used for moisturizing skin, and in particular, a more superior moisturizing effect is observed when two or more mixed extracts are used.

Specifically, the present invention may provide a cosmetic composition for improving skin troubles including one or more extracts selected from the group consisting of Dimocarpus longan Fruit, Vitex trifolia Fruit and Aquilaria agallocha Stem, or a fraction thereof, as an active ingredient.

As used herein, the term “skin troubles” refer to a skin disorder that may be caused by inflammation due to excessive nitric oxide production in macrophages. The skin troubles include all kinds of skin diseases related to inflammation regardless of the types. Non-limiting examples of the skin diseases related to inflammation include erythema caused by atopic dermatitis, psoriasis, radiation, chemical substances, burns, and the like, acid burns, bullous dermatosis, lichenoid dermatitis, itching caused by allergy, seborrheic eczema, rosacea, pemphigus vulgaris, erythema exsudativum multiforme, erythema nodosum, balanitis, vulvitis, inflammatory hair loss such as alopecia areata, skin T-cell lymphoma and the like, and specifically include skin rash, acne, pimples, rosacea (red nose), but are not limited thereto. Further, as used herein, the term “improvement of skin troubles” refers to inhibiting or suppressing the formation of troubles on the skin, or alleviating skin troubles that have already been formed.

As used herein, the term “anti-inflammation” refers to inhibition of inflammation, and the inflammation, which is one of defensive responses of biological tissues to any stimuli, and refers to a complicated lesion which develops three types of tissue degeneration, circulatory disturbance and exudation, and tissue proliferation. More specifically, inflammation is a part of innate immunity and, human innate immunity recognizes a pattern on the cell surfaces that are specifically present in a pathogen, like in other animals. Phagocytes recognize cells with such surfaces as non-self and attack pathogens. If pathogens break and penetrate physical barriers of the body, an inflammation reaction occurs. The inflammation reaction is a non-specific defense that creates an environment hostile to microbes at the site of injury. In an inflammatory reaction, when injury occurs or pathogens invade the body, cytokines are secreted by the recruitment of leukocytes involved in initial immune responses. Therefore, the intracellular cytokine level is an index of inflammatory activation. The anti-inflammation of the present invention may inhibit inflammation occurred in the skin.

In one embodiment of the present invention, NO production inhibitory effect of one or more extracts selected from the group consisting of Dimocarpus longan Fruit, Vitex trifolia Fruit and Aquilaria agallocha Stem was measured, and as a result, all the experimental groups to which the extract was added exhibited a superior NO production inhibitory effect compared to the control group, and when two mixed extracts were added, the NO production inhibitory effect was further enhanced compared to the case where a single extract was treated, and in particular, the highest NO production inhibitory effect was obtained when three mixed extracts were treated (Table 6).

The Dimocarpus longan Fruit, Vitex trifolia Fruit and Aquilaria agallocha Stem extracts of the present invention have an anti-inflammatory effect through the NO production inhibitory effect, and thus can be used for improving skin troubles. In particular, a more superior trouble-improving effect is observed when two or more mixed extracts are used.

In conclusion, the Dimocarpus longan Fruit, Vitex trifolia Fruit and Aquilaria agallocha Stem extracts of the present invention have the effects of enhancing skin elasticity or improving wrinkles, moisturizing skin, or improving skin troubles, when treated alone. Further, the extracts in which two or more selected from the group consisting of Dimocarpus longan Fruit, Vitex trifolia Fruit and Aquilaria agallocha Stem are mixed exhibit excellent effects in enhancing skin elasticity or improving wrinkles, moisturizing skin, or improving skin troubles, as compared with the case where each extract is used alone. The synergistic effect of the mixed extracts of two or more selected from the group consisting of Dimocarpus longan Fruit, Vitex trifolia Fruit and Aquilaria agallocha Stem is enhanced by the synergistic effect or cooperative action between the extracts and/or between the active ingredients in the extracts, thereby exhibiting more superior efficacy in enhancing skin elasticity or improving wrinkle, moisturizing skin, or improving skin trouble, as compared to each single extract, and such findings were first identified in the present invention.

The composition may be prepared into a formulation selected from the group consisting of solution, topical ointment, cream, foam, nutritive cosmetic water, softening cosmetic water, pack, softening water, latex, makeup base, essence, soap, liquid washing agent, bath foam, sunscreen cream, sun oil, suspension, emulsion, pastes, gel, lotion, powder, soap, surfactant-containing cleanser, oil, powder foundation, emulsion foundation, wax foundation, patch and spray, but is not limited thereto.

Further, the cosmetic composition of the present invention may further include at least one cosmetically acceptable carrier mixed to a general skin cosmetic composition. As common ingredients, for example, oil, water, surfactants, moisturizers, lower alcohols, thickening agents, chelating agents, colorings, preservatives, fragrances, etc. may be appropriately mixed, but are not limited thereto.

The cosmetically acceptable carrier contained in the cosmetic composition of the present invention may vary depending on the formulations.

When the formulation of the present invention is a paste, cream or gel, animal oil, vegetable oil, wax, paraffin, starch, tragacanth, cellulose derivatives, polyethylene glycol, silicone, bentonite, silica, talc, zinc oxide, or mixtures thereof may can be used as a carrier ingredient.

When the formulation of the present invention is a powder or spray, lactose, talc, silica, aluminum hydroxide, calcium silicate, polyamide powder or mixtures thereof may be used as a carrier ingredient, and in particular, when it is a spray, a propellant such as chlorofluorohydrocarbon, propane/butane or dimethyl ether may be additionally included.

When the formulation of the present invention is a solution or turbid fluid, solvents, solubilizing agents or emulsifying agents may be used as a carrier ingredient, and for example, water, ethanol, isopropanol, ethyl carbonate, ethyl acetate, benzyl alcohol, benzyl benzoate, propylene glycol, 1,3-buthylglycol oil may be used. In particular, cottonseed oil, peanut oil, maize germ oil, olive oil, castor oil and sesame seed oil, glycerol aliphatic ester, polyethylene glycol or aliphatic ester of sorbitan may be used.

When the formulation of the present invention is a suspension, liquid diluents such as water, ethanol or propylene glycol, suspending agent such as ethoxylated isostearyl alcohol, polyoxyethylene sorbitol ester and polyoxyethylene sorbitan ester, microcrystalline cellulose, aluminum metahydroxide, bentonite, agar, or tragacant, and the like may be used as a carrier ingredient.

When the formulation of the present invention is a soap, alkali metal salts of fatty acids, fatty acid hemiester salts, fatty acid protein hydrolysates, isethionate, lanolin derivatives, aliphatic alcohols, vegetable oil, glycerol, sugars and the like may be used as a carrier ingredient.

In the cosmetic composition of the present invention, one or more extracts selected from the group consisting of Dimocarpus longan Fruit, Vitex trifolia Fruit and Aquilaria agallocha Stem may be specifically contained in an amount of 0.0001% to 50% by weight, more specifically 0.0005% to 10% by weight on a dry weight based on the total weight of the cosmetic composition. The above-mentioned content range exhibits excellent skin elasticity enhancement or wrinkle improvement; skin moisturization; or skin trouble improvement efficacy and may be adjusted according to the requirements such as stabilization of the formulation of the composition.

In another aspect, the present invention provides a food composition including one or more extracts selected from the group consisting of Dimocarpus longan Fruit, Vitex trifolia Fruit and Aquilaria agallocha Stem, or a fraction thereof, as an active ingredient.

Specifically, the present invention may provide a food composition for enhancing skin elasticity or improving wrinkles; moisturizing skin; or improving skin troubles including one or more extracts selected from the group consisting of Dimocarpus longan Fruit, Vitex trifolia Fruit and Aquilaria agallocha Stem, or a fraction thereof, as an active ingredient.

The Dimocarpus longan Fruit extract, Vitex trifolia Fruit extract, and Aquilaria agallocha Stem extract, skin elasticity enhancement, wrinkle improvement, skin moisturization, and skin trouble improvement are as described above. The food composition may be used in the form of a health functional food, but is not limited thereto.

One or more extracts selected from the group consisting of Dimocarpus longan Fruit, Vitex trifolia Fruit and Aquilaria agallocha Stem included in the food composition of the present invention may be contained in the form of an animal or plant containing the extract of the present invention, an extract thereof, a fraction thereof or a processed product thereof. In addition, the composition may also include food acceptable food supplement additives in addition to the active ingredients.

As used herein, the term “food supplement additives” refers to constituent elements which can be adjunctly added to foods, and are added when producing health functional foods of each formulation and can be appropriately selected and used by those skilled in the art. Examples of food supplement additives may include various nutrients, vitamins, minerals (electrolytes), flavor agents such as synthetic flavor agents, natural flavor agents, and the like, coloring agents, fillers, pectic acid and salts thereof, alginic acid and salts thereof, organic acids, protective colloid thickeners, PH adjusters, stabilizers, preservatives, glycerin, alcohols, carbonating agents used for a carbonated drink and the like, but the types of food supplement additives are not limited thereto.

The food composition of the present invention may include a health functional food. As used herein, the term “health functional food” refers to a food prepared and processed in the form of tablets, capsules, powder, granules, liquid and pills by using raw materials or ingredients having useful functionality to the human body. As used herein, the term “functionality” refers to controlling nutrients for the structure of functions of the human body or providing useful effects for hygienic purposes, such as psychological effects, and the like. The health functional food of the present invention may be prepared using methods commonly used in the art, and may be prepared by adding raw materials and ingredients commonly added in the art. In addition, the formulation of the health functional food is not particularly limited so long as it is recognized as a health functional food. The food composition of the present invention may be prepared in various formulations, and the food composition of the present invention uses a food as a raw material unlike generic drugs, and thus has no side effects that may occur during long-term administration thereof, is highly portable, and may be administered as an adjuvant for enhancing the effects of enhancing skin elasticity or improving wrinkles, moisturizing skin, or improving skin troubles.

There is no limitation on the form that the health functional food of the present invention can take, and it can include all foods in the common meaning, and can be used interchangeably with terms known in the art such as functional foods. In addition, the health functional food of the present invention may be prepared by mixing with other suitable supplement ingredients and additives known in the art that can be included in the foods, depending on the selection by those skilled in the art. Examples of the foods to which it can be added include meats, sausages, bread, chocolates, candies, snacks, confectionaries, pizzas, instant noodles, other noodles, gums, dairy products including ice creams, various kinds of soup, beverages, teas, drinks, alcoholic drinks, vitamin complexes, and the like. Further, the health functional food of the present invention may be prepared by adding it to concentrates, teas, jellies, and juice prepared with the extract of the present invention as the main ingredient. In addition, foods used as feed for animals may be included.

In still another aspect, the present invention provides a quasi-drug composition including one or more extracts selected from the group consisting of Dimocarpus longan Fruit, Vitex trifolia Fruit and Aquilaria agallocha Stem, or a fraction thereof, as an active ingredient.

Specifically, the present invention may provide a quasi-drug composition for enhancing skin elasticity or improving wrinkles; moisturizing skin; or improving skin trouble including one or more extracts selected from the group consisting of Dimocarpus longan Fruit, Vitex trifolia Fruit and Aquilaria agallocha Stem, or a fraction thereof, as an active ingredient.

The Dimocarpus longan Fruit extract, Vitex trifolia Fruit extract, and Aquilaria agallocha Stem extract, skin elasticity enhancement, wrinkle improvement, skin moisturization, and skin trouble improvement are as described above.

The quasi-drug composition of the present invention may further contain a pharmaceutically acceptable carrier, excipient or diluent as necessary in addition to the above components. The pharmaceutically acceptable carrier, excipient or diluent is not limited as long as the effect of the present invention is not impaired, and may include, for example, fillers, extenders, binders, wetting agents, disintegrants, surfactants, lubricants, sweeteners, flavoring agents, preservatives, and the like.

Typical examples of the pharmaceutically acceptable carrier, excipient, and diluent of the present invention include lactose, dextrose, sucrose, sorbitol, mannitol, xylitol, maltitol, starch, gelatin, glycerin, acacia gum, alginate, calcium phosphate, calcium carbonate, calcium silicate, cellulose, methyl cellulose, microcrystalline cellulose, polyvinyl pyrrolidone, water, methyl hydroxybenzoate, propyl hydroxybeonzoate, talc, magnesium stearate, mineral oil, propylene glycol, polyethylene glycol, vegetable oil, injectable ester, witepsol, macrogol, tween 61, cacao butter, laurin butter and the like.

In addition, when the extract of the present invention is used as a quasi-drug, it may further contain one or more active ingredients exhibiting the same or similar functions. For example, it may contain an elasticity enhancing ingredient, wrinkle improving ingredient, moisturizing ingredient, or anti-inflammatory ingredient known in the art. When an additional elasticity enhancing ingredient, wrinkle improving ingredient, moisturizing ingredient, or anti-inflammatory ingredient is contained, the skin elasticity enhancing effect, wrinkle improving effect, moisturizing effect, or skin trouble improving effect of the composition of the present invention may be further enhanced. When the above additional ingredients are added, skin safety, easiness of formulation, and stability of effective ingredients may be considered according to the combined use. In addition, the quasi-drug composition may further include one or two or more ingredients selected from the group consisting of retinoic acid, TGF, an animal placenta-derived protein, betulinic acid, and a chlorella extract, as an elasticity enhancing ingredient, wrinkle-improving ingredient or moisturizing ingredient known in the art; non-steroid such as flufenamic acid, ibuprofen, benzydamine, indomethacin, and steroid such as prednisolone, dexamethasone, allantoin, azulene, hydrocortisone as an anti-inflammatory ingredient known in the art; and derivatives thereof and various plant extracts. The additional ingredients may be contained in an amount of 0.0001 to 10% by weight based on the total weight of the composition. The content range may be adjusted according to the requirements such as skin safety, easiness when formulating Dimocarpus longan Fruit, Vitex trifolia Fruit and Aquilaria agallocha Stem extracts or mixed extracts thereof, and the like.

Examples of the quasi-drug composition of the present invention may include disinfecting cleaners, shower foams, ointments, wet wipes, coating agents, and the like, but are not limited thereto. The formulation method, dosage, method of use, constituent components, and the like of the quasi-drug can be suitably selected from conventional techniques known in the art

Moreover, the quasi-drug composition may be used in a method for enhancing skin elasticity or improving wrinkles; moisturizing skin; or improving skin troubles, including applying one or more extracts selected from the group consisting of Dimocarpus longan Fruit, Vitex trifolia Fruit and Aquilaria agallocha Stem of the present invention to the skin of an individual. The individual includes, without limitation, mammals including rats, livestock, humans, and the like.

### MODE FOR CARRYING OUT THE INVENTION

Hereinafter, the constitution and effect of the present invention will be described by way of specific Examples. However, these Examples are given for illustrative purposes only, and the scope of the invention is not intended to be limited by these Examples.

### PREPARATION EXAMPLE 1

### Preparation of Dimocarpus longan Fruit, Vitex trifolia Fruit and/or Aquilaria agallocha Stem Extract

After drying finely cutting Dimocarpus longan Fruit, Vitex trifolia Fruit and Aquilaria agallocha Stem, 100 g of each herb on a dry weight was put into a flask and extracted with 1,000 g of an extraction solvent (distilled water) for 3 days via cold-water extraction. The cold-water extract was filtered with a filter having a pore size of 0.2 μm to prepare each of Dimocarpus longan Fruit, Vitex trifolia Fruit and Aquilaria agallocha Stem extracts.

In addition, mixed extracts were prepared by mixing each of the Dimocarpus longan Fruit extract, Vitex trifolia Fruit extract, and Aquilaria agallocha Stem extract prepared in Preparation Example 1. Production ratios of the mixed extracts are shown in Table 1.

TABLE 1

| Ingredients | Example 1 | Example 2 | Example 3 | Example 4 | Example 5 | Example 6 | Example 7 |
|---|---|---|---|---|---|---|---|
| Dimocarpus Longan | 3 | | | 1.5 | 1.5 | | 1 |
| Fruit | | | | | | | |
| Vitex Trifolia | | 3 | | 1.5 | | 1.5 | 1 |
| Fruit | | | | | | | |
| Aquilaria Agallocha | | | 3 | | 1.5 | 1.5 | 1 |
| Stem | | | | | | | |

### PREPARATION EXAMPLE 2

### Preparation of Essence Sontaining Dimocarpus longan Fruit, Vitex trifolia Fruit and/or Aquilaria agallocha Stem Extract

Essence containing the extracts of Examples 1 to 7 was prepared. The detailed composition (% by weight) constituting the essence is shown in Table 2 below.

TABLE 2

| | Essence of | |
|---|---|---|
| | the present | Comparative |
| Essence Composition | invention | Example |
| Dimocarpus Longan Fruit, Vitex | 3 | — |
| Trifolia Fruit and/or Aquilaria | | |
| Agallocha Stem Extract | | |
| Triethanolamine | 0.25 | 0.25 |
| Carboxyvinyl polymer (1) | 0.22 | 0.22 |
| Glycerin | 4 | 4 |
| Propylene glycol | 0.2 | 0.2 |
| p-hydroxybenzoate Ester | 0.2 | 0.2 |
| Beeswax | 0.5 | 0.5 |
| Cetostearyl alcohol | 1 | 1 |
| Glyceryl monostearate | 1 | 1 |
| Sorbitan monostearate (2) | 0.5 | 0.5 |
| Polyethylene glycol monostearate | 8 | 8 |
| Propyl p-hydroxybenzoate | 0.1 | 0.1 |
| Dimethylsiloxane | 0.3 | 0.3 |
| Isocetyl octanoate (3) | 3 | 3 |
| Squalane | 5 | 5 |
| Fragrance | Suitable | Suitable |
| | amount | amount |
| Distilled water | Residual | Residual |
| | amount | amount |

(1). Carboxyvinyl polymer - Carbopol 941 1 wt % aqueous solution manufactured by B.F. Goodrich, USA
(2). Sorbitan monostearate - Arlacel 60 manufactured by ICI, England
(3). Isocetyl octanoate - ICEH manufactured by Nihon KoKyu Alcohol, Japan

### EXPERIMENTAL EXAMPLE 1

### Effect of Promoting Type 1 Collagen Synthesis in Human Fibroblasts—Effect of Enhancing Skin Elasticity or Improving Wrinkles

Seven kinds of extracts obtained in Preparation Example 1 were added to culture media of human-derived fibroblasts in order to confirm the effect of promoting type I collagen synthesis at the cellular level. The synthesized collagen was measured and quantitated using a PICP EIA kit (Procollagen Type I C-Peptide Enzyme Immuno Assay Kit). In order to measure the amount of synthesized collagen, the extracts of Examples 1 to 7 were added to the culture media (DMEM medium) of the fibroblasts at final concentrations of 1,000 ppm and 10,000 ppm, and cultured for 48 hours. The culture broth was collected and the degree of type 1 collagen synthesis was measured with a PICP EIA kit at 450 nm using a spectrophotometer.

For the comparison of the effects, the degree of collagen synthesis was measured in the same manner for the culture medium of the fibroblasts not treated with the extract (negative control) and the culture medium (positive control) in which vitamin C was added to a final concentration of 52.85 ppm. The rate of increase in collagen production was calculated as a ratio of collagen production relative to negative control, and the results are shown in Table 3 below. The experiments were conducted four times and the results are expressed as an average value.

TABLE 3

| | Production amount of type 1 | Increased |
|---|---|---|
| Samples | collagen (ng/ml) | rate (%) |
| Negative Control | 150.2 | — |
| Positive Control (Vitamin C, | 246.8 | 64.3 |
| 52.85 ppm) | | |
| Example 1 (1000 ppm) | 181.1 | 20.6 |
| Example 1 (10000 ppm) | 220.3 | 46.7 |
| Example 2 (1000 ppm) | 175.4 | 16.8 |
| Example 2 (10000 ppm) | 210.5 | 40.1 |
| Example 3 (1000 ppm) | 172.8 | 15.0 |
| Example 3 (10000 ppm) | 208.7 | 38.9 |
| Example 4 (1000 ppm) | 200.2 | 33.3 |
| Example 4 (10000 ppm) | 240.1 | 59.9 |
| Example 5 (1000 ppm) | 205.7 | 37.0 |
| Example 5 (10000 ppm) | 237.4 | 58.1 |
| Example 6 (1000 ppm) | 201.8 | 34.4 |
| Example 6 (10000 ppm) | 235.8 | 57.0 |
| Example 7 (1000 ppm) | 240.8 | 60.3 |
| Example 7 (10000 ppm) | 266.6 | 77.5 |

(Repeated # = 4)

As can be seen from the results in Table 3, it was confirmed that when the extracts of Examples 1 to 3 were treated, which were single extracts of Dimocarpus longan Fruit, Vitex trifolia Fruit and Aquilaria agallocha Stem, the collagen synthesis was increased in all cases as compared with the negative control, and such an increase was concentration dependent. In addition, when the extracts of Examples 4 to 6, which were two mixed extracts, the effect of collagen synthesis was further enhanced as compared to Examples 1 to 3 in which the single extracts were treated at the same concentration. In particular, it was confirmed that the highest collagen synthesis effect was observed in Example 7 in which all extracts of Dimocarpus longan Fruit, Vitex trifolia Fruit and Aquilaria agallocha Stem were mixed.

From this, it was found that each of the Dimocarpus longan Fruit, Vitex trifolia Fruit and Aquilaria agallocha Stem extracts had a collagen synthesis effect, and the effect was further enhanced when the extracts were used as mixtures. Since the extracts of Examples 1 to 7 induce collagen synthesis, they can be used for enhancing skin elasticity, improving wrinkles or moisturizing skin.

### EXPERIMENTAL EXAMPLE 2

### Inhibitory Effect of Elastase Activity—Effects of Enhancing Skin Elasticity or Improving Wrinkle, Moisturizing Skin

It was confirmed whether the seven kinds of extracts obtained in Preparation Example 1 had an effect of inhibiting elastase activity, which is an enzyme that degrades elastin.

Elastase derived from human leukocyte cells was used, and MeOSuc-Ala-Ala-Pro-Val-pNA, a synthetic substrate, was used as a substrate for elastase. 100 mM Tris (pH 7.5) solution was used as a buffer solution. Elastase was used at a final concentration of 0.2 mU using a buffer solution. Further, the synthetic substrate of elastase was prepared into a 100 mM solution using DMSO and then diluted with the buffer solution to give a final concentration of 0.5 mM. At this time, the positive control group was prepared by adding 10 ppm of quercetin, which is known as an elastase inhibitor. The extracts of Examples 1 to 7 were added to final concentrations of 1,000 ppm and 10,000 ppm. The reaction was carried out in a 96-well plate and allowed to react at room temperature for 20 minutes. Absorbance was measured at 405 nm using a spectrophotometer at 1-minute intervals to calculate the slope of the absorbance relative to time, and the activity of the enzyme was measured. The inhibition rate of elastase was calculated as shown in the following Calculation Equation 1, and the results are shown in Table 4 below. The experiments were each conducted three times and the results are expressed as an average value

Inhibition Rate of Elastase (%)=(Slope of Control−Slope of Sample)/Slope of Control×100 8 Calculation Equation 1]

TABLE 4

| | | Inhibition Rate |
|---|---|---|
| Samples | Enzyme Activity | (%) |
| Control (DMSO, 20 ppm) | 10.4 | — |
| Positive Control (Quercetin, | 3.5 | 66.3 |
| 10 ppm) | | |
| Example 1 (1000 ppm) | 4.6 | 55.8 |
| Example 1 (10000 ppm) | 2.9 | 72.1 |
| Example 2 (1000 ppm) | 4.5 | 56.7 |
| Example 2 (10000 ppm) | 3.1 | 70.2 |
| Example 3 (1000 ppm) | 4.6 | 55.8 |
| Example 3 (10000 ppm) | 3.1 | 70.2 |
| Example 4 (1000 ppm) | 4.2 | 59.6 |
| Example 4 (10000 ppm) | 2.5 | 76.0 |
| Example 5 (1000 ppm) | 4.2 | 59.6 |
| Example 5 (10000 ppm) | 2.3 | 77.9 |
| Example 6 (1000 ppm) | 4.1 | 60.6 |
| Example 6 (10000 ppm) | 2.2 | 78.8 |
| Example 7 (1000 ppm) | 3.6 | 65.4 |
| Example 7 (10000 ppm) | 1.8 | 82.7 |

(Repeated # = 3)

As can be seen from the results in Table 4, it was confirmed that when the extracts of Examples 1 to 3 were treated, which were single extracts of Dimocarpus longan Fruit, Vitex trifolia Fruit and Aquilaria agallocha Stem, the inhibitory effect of elastase activity was observed in a concentration-dependent manner in all cases. In addition, when the extracts of Examples 4 to 6, which were two mixed extracts, the inhibitory effect of elastase activity was further enhanced as compared to Examples 1 to 3 in which the single extracts were treated at the same concentration. In particular, it was confirmed that the highest inhibitory effect of elastase activity was observed in Example 7 in which all extracts of Dimocarpus longan Fruit, Vitex trifolia Fruit and Aquilaria agallocha Stem were mixed.

From this, it was found that each of the Dimocarpus longan Fruit, Vitex trifolia Fruit and Aquilaria agallocha Stem extracts exhibited an inhibitory effect of elastase activity, and the effect was further enhanced when the extracts were used as mixtures. Since the extracts of Examples 1 to 7 inhibit elastase activity, they can be used for enhancing skin elasticity, improving wrinkles or moisturizing skin.

### EXPERIMENTAL EXAMPLE 3

### Evolution of Moisture Retention Capacity—Skin Moisturizing Effect

In order to evaluate the skin moisturizing power of the essence containing Dimocarpus longan Fruit, Vitex trifolia Fruit and Aquilaria agallocha Stem extracts prepared in Preparation Example 2, each essence was applied to the skin, and then the moisture retention amount in the stratum corneum was measured and evaluated. As a measuring device, a skin moisture analyzer (skin surface hygrometer, model name: SKICON-200, manufactured by IBS, Japan) was used.

Specifically, a certain amount (2 mg/cm²) of the essence was applied to the inner arms of each of 20 subjects and rubbed well in a constant temperature and humidity room at 25° C. and a relative humidity of 45. The rate of increase in moisture (%) was measured after 1 hour and 3 hours relative to the moisture retention capacity before evaluation to evaluate the moisturizing effect. The obtained results are shown in Table 5 below.

TABLE 5

| | Rate of increase in moisture (%) | | | | | | | |
|---|---|---|---|---|---|---|---|---|
| | | | | | | | | Dimocarpus Longan |
| | | | | | | | | Fruit + Vitex |
| | Compar- | | Vitex | Aquilaria | Dimocarpus Longan | Dimocarpus Longan | Vitex Trifolia | Trifolia Fruit + |
| | ative | Dimocarpus | Trifolia | Agallocha | Fruit + Vitex | Fruit + Aquilaria | Fruit + Aquilaria | Aquilaria Agallocha |
| Category | Example | Longan Fruit | Fruit | Stem | Trifolia Fruit | Agallocha Stem | Agallocha Stem | Stem |
| Before | 0 | 0 | 0 | 0 | 0 | 0 | 0 | 0 |
| application | | | | | | | | |
| After | 175 | 224 | 220 | 221 | 232 | 233 | 235 | 261 |
| 1 hour of | | | | | | | | |
| application | | | | | | | | |
| After | 160 | 189 | 180 | 167 | 210 | 200 | 201 | 243 |
| 3 hours of | | | | | | | | |
| application | | | | | | | | |

As can be seen from the results of Table 5, in the case of the essence containing the single extracts of Dimocarpus longan Fruit, Vitex trifolia Fruit and/or Aquilaria agallocha Stem, the rate of increase in moisture after 1 hour and 3 hours of the application was higher in all cases as compared with the Comparative Example not containing the extract. In addition, when the essence containing the two mixed extracts was applied, the rate of increase in moisture was further increased. In particular, it was confirmed that the highest rate of increase in moisture was observed after 1 hour and 3 hours of the application, when the essence containing three mixed extracts including all of the Dimocarpus longan Fruit, Vitex trifolia Fruit and Aquilaria agallocha Stem extracts was used.

From this, it was found that each of the Dimocarpus longan Fruit, Vitex trifolia Fruit and Aquilaria agallocha Stem extracts had a skin moisturizing effect, and the effect was further enhanced when the extracts were used as mixtures. Accordingly, one or more extracts selected from the group consisting of Dimocarpus longan Fruit, Vitex trifolia Fruit and Aquilaria agallocha Stem have excellent moisture retention power and thus can be used in skin moisturization compositions.

### EXPERIMENTAL EXAMPLE 4

### Anti-Inflammatory Effect by Inhibition of NO Production—Skin Trouble Improving Effect

In order to confirm the anti-inflammatory effect and the skin trouble-improving effect of the seven extracts obtained in Preparation Example 1, an experiment on the inhibition of nitrogen monoxide (NO) formation was conducted by the GRIESS method using RAW264.7 cell line (ATCC number: CRL-2278).

Specifically, RAW264.7 cells, which were macrophages of mice, were subcultured several times, and the cultured cells were put into a 24-well plate at a density of 3×10⁵ per cell, and cultured for 24 hours. Subsequently, the cells were replaced with cell culture media containing the above-mentioned 7 kinds of extracts diluted to concentrations of 100 ppm, 100 ppm and 100 ppm. At this time, L-NMMA (L-NG-monomethylarginine), a NO production inhibitor, was treated together as a positive control and cultured for 30 minutes. 1 μg of LPS (lipopolysaccharide) was treated each time as a stimulant and cultured for 24 hours. 100 μL of the supernatant was collected and transferred to a 96-well plate, and 100 μL of GRIESS solution was added thereto and reacted at room temperature for 10 minutes, and then the absorbance at 540 nm was measured to determine the NO inhibitory effect of the extracts. The NO production inhibition rate (%) was calculated using the following Calculation Equation 2, and the results are shown in Table 6. The experiments were each carried out three times, and the results are expressed as an average value.

Inhibition Rate of NO Production (%)={(Absorbance of negative control−Absorbance of each extract)/Absorbance of negative control}×100 [Calculation Equation 2]

TABLE 6

| | Inhibition Rate of NO |
|---|---|
| Samples | Production (%) |
| Negative control (DMSO, 10 ppm) | — |
| Positive control (L-NMMA, | 41.0 |
| 10 ppm) | |
| Example 1 (100 ppm) | 13.1 |
| Example 1 (1000 ppm) | 15.9 |
| Example 1 (10000 ppm) | 40.1 |
| Example 2 (100 ppm) | 12.5 |
| Example 2 (1000 ppm) | 14.2 |
| Example 2 (10000 ppm) | 38.3 |
| Example 3 (100 ppm) | 12.6 |
| Example 3 (1000 ppm) | 15.1 |
| Example 3 (10000 ppm) | 39.9 |
| Example 4 (100 ppm) | 15.1 |
| Example 4 (1000 ppm) | 20.1 |
| Example 4 (10000 ppm) | 50.5 |
| Example 5 (100 ppm) | 16.1 |
| Example 5 (1000 ppm) | 20.8 |
| Example 5 (10000 ppm) | 53.1 |
| Example 6 (100 ppm) | 16.1 |
| Example 6 (1000 ppm) | 21.0 |
| Example 6 (10000 ppm) | 50.1 |
| Example 7 (100 ppm) | 17.5 |
| Example 7 (1000 ppm) | 22.6 |
| Example 7 (10000 ppm) | 59.8 |

(Repeated # = 3)

As can be seen from the results in Table 6, it was confirmed that when the extracts of Dimocarpus longan Fruit, Vitex trifolia Fruit and Aquilaria agallocha Stem of Examples 1 to 3 were each treated, the NO production inhibitory effect was observed in a concentration-dependent manner. In addition, when the extracts of Examples 4 to 6, which were two mixed extracts, were treated, the NO production inhibitory effect was further enhanced as compared to Examples 1 to 3 in which the single extracts were treated at the same concentration. In particular, it was confirmed that the highest NO production inhibitory effect was observed in Example 7 in which all extracts of Dimocarpus longan Fruit, Vitex trifolia Fruit and Aquilaria agallocha Stem were mixed.

From this, it was found that each of the Dimocarpus longan Fruit, Vitex trifolia Fruit and Aquilaria agallocha Stem extracts exhibited an NO production inhibitory effect, and the effect was further enhanced when the extracts were used as mixtures. Since the extracts of Examples 1 to 7 exhibit an anti-inflammatory effect through the inhibition of NO production, they can be used for improving skin troubles, and also can play an auxiliary role in skin whitening, elasticity enhancement, wrinkles improvement and skin moisturization, and thus, a synergy effect can be expected.

1. A cosmetic composition comprising one or more extracts selected from the group consisting of Dimocarpus longan Fruit, Vitex trifolia Fruit and Aquilaria agallocha Stem, ef-and a fraction thereof, as an active ingredient.

2. The cosmetic composition of claim 1, wherein the composition is for enhancing skin elasticity or improving wrinkles.

3. The cosmetic composition of claim 1, wherein the composition is for moisturizing skin.

4. The cosmetic composition of claim 1, wherein the composition is for improving skin troubles.

5. The cosmetic composition of claim 1, wherein the composition has a collagen synthesis-promoting, elastase activity-inhibiting, moisturizing, or anti-inflammatory effect.

6. The cosmetic composition of claim 1, wherein the composition is in a form of a formulation selected from the group consisting of solution, topical ointment, cream, foam, nutritive cosmetic water, softening cosmetic water, pack, softening water, latex, makeup base, essence, soap, liquid washing agent, bath foam, sunscreen cream, sun oil, suspension, emulsion, paste, gel, lotion, powder, soap, surfactant-containing cleanser, oil, powder foundation, emulsion foundation, wax foundation, patch, and spray.

7. A food composition comprising one or more extracts selected from the group consisting of Dimocarpus longan Fruit, Vitex trifolia Fruit and Aquilaria agallocha Stem, and a fraction thereof, as an active ingredient.

8. The food composition of claim 7, wherein the food composition is for enhancing skin elasticity or improving wrinkles; moisturizing skin; or improving skin troubles.

9. A quasi-drug composition comprising one or more extracts selected from the group consisting of Dimocarpus longan Fruit, Vitex trifolia Fruit and Aquilaria agallocha Stem, and a fraction thereof, as an active ingredient.

10. The quasi-drug composition of claim 9, wherein the quasi drug composition is for enhancing skin elasticity or improving wrinkles; moisturizing skin; or improving skin troubles.
